(19) United States
(12) Patent Application Publication
(10) Pub. No.: US 2013/0261420 A1
(43) Pub. Date: Oct. 3, 2013

(54) System and method for non-invasive diagnostic of mammals

(71) Applicants: Semen Kucherov, Moscow (RU); Valentin Barzinsky, Kiev (UA); Irina Kucherova, Moscow (RU); Sergey Marchenko, Moscow (RU)

(72) Inventors: Semen Kucherov, Moscow (RU); Valentin Barzinsky, Kiev (UA); Irina Kucherova, Moscow (RU); Sergey Marchenko, Moscow (RU)

(21) Appl. No.: 13/134,356

(22) Filed: Jun. 6, 2011

(51) Int. Cl. A61B 5/04

(52) U.S. Cl. 600/372

(57) ABSTRACT

An apparatus of the present invention is used for a non-invasive diagnostics and correction of humans and animals. The apparatus includes a computer, a reference database of the computer. A reversible sensing electrode device adaptable for receiving information about biological active cells of the human as the information is received through spectral signals. The audio processor receives the spectral signals from the reversible sensing electrode device and transforming the spectral signals to the digital codes. The audio processor is cooperable with the reversible sensing electrode device for receiving the spectral signals therefrom and transforming the spectral signals to digital codes.

### FIELD OF THE INVENTION

The present invention relates to a system for medical diagnosis of disease in a mammal, and more particularly to a system and method for non-invasive diagnostics of a human body.

### BACKGROUND OF THE INVENTION

Today, computer diagnostic methods and systems are considered to be the most widely used means for diagnosing the general state of a human body as well as of its separate organs and systems. Various complex computer diagnosing systems (CDS) attract the attention of health professionals, and particularly patients, first of all, by its comfort, quick results, their variety and colourful presentation, and, moreover, by non-invasive technique, thereby eliminating need for a patient to take internal medicine, such as special pills, or test drugs. Moreover, a patient has no need to needle fingers and veins, take a blood test, to be afraid of blood-poisoning by dangerous infections and incurable diseases.

These computer diagnostic methods, also known as non-invasive diagnostics of human body, became more popular over the recent years. These types of diagnostics are used to determine whether there is onset of disease in the human body because the decease of any kind is usually accompanied by physical changes in a human body. Some physical changes, while not known to a human, can be detected with appropriate diagnostic equipment, often at a relatively early stage of the disease.

Some types of these equipments and methods require invasive procedures to determine if the body is affected by the onset. Other types of the diagnostic methods require the human to take medicine internally, which may affect negative other organs of the human or if the human has certain allergies to the medicine.

Those skilled in the art will appreciate that a decrease in electrical impedance occurs in tissue as it undergoes cancerous changes. Consequently, electrical impedance may be used to diagnose disease. A method that permits comparisons of electrical properties for diagnostic purposes has been developed that involves homologous body parts, i.e., body parts that are substantially similar, such as a left breast and a right breast. In this method, the impedance of a body part of a patient is compared to the impedance of the homologous body part of the same patient. However, this method is not always accurate and may result in situation where certain life threatening conditions of the human may not be noticed.

The art is replete with prior art systems and methods of non-invasive diagnostics. U.S. Pat. No. 3,971,366 to Motoyama teaches an apparatus which diagnoses the functional condition of every internal organ and the balance condition of the autonomic nervous system in a living body has a reference electrode firmly attached to a part of the living body, a plurality of differential electrodes firmly attached to each of specific minute points which are located symmetrically in the tips of the members, i.e. fingers of a living body, a detection circuit to which the electrodes are connected to detect electric resistance in the surface layer of the living body generated between the reference electrode and the respective differential electrodes, an average value measurement circuit connected to the detecting circuit to obtain an average value from the detected output and a maximum value measurement circuit connected to the detection circuit to obtain a maximum value from said output. The apparatus diagnoses the function of every internal organ by measuring the change of electric resistance at a plurality of specific points in the surface layer of the living body which are directly related to the internal organ. The apparatus disclosed in the U.S. Pat. No. 3,971,366 to Motoyama requires numerous electrodes to be attached to the specific points on the fingers of the human, which is time consuming procedure that requires multitude of elements and exact location on the fingers to be symmetrically positioned in the tips of the fingers. Moreover, this apparatus is impracticable where the human has no finger or parts of the fingers.

Another prior art reference, i.e. U.S. Pat. No. 4,794,934 to Motoyama teaches an apparatus for diagnosing the functions of the internal organs and the autonomic nervous system of the living body in which a constant voltage is applied across two or more electrodes attached to the living body, a current flowing across the electrodes and changing from time to time is detected at high speed and recorded, and predetermined parameters are calculated from the recorded data is disclosed. The electrodes are sequentially or arbitrarily scanned by an electrode scanning means, and the constant voltage generated by a constant voltage generating means is applied to the scanned electrodes. The instantaneous value of the current flowing across two electrodes is detected by a detecting means, and recorded as a digital value in a recording means. The data recorded in the recording means are displayed on a display means via a transmitting means, and/or the parameters calculated by a calculating means from the data recorded in the recording means are displayed on the display means. The apparatus taught by the U.S. Pat. No. 4,794,934 to Motoyama is limited for diagnosing the functions of the internal organs and the autonomic nervous system only and does not provide means for determining condition of other parts of the human thereby limiting the functionality and use of the apparatus.

Another prior art technique for screening and diagnosing diseased states within the body using electrical impedance is disclosed in U.S. Pat. No. 6,122,544 to Organ. This prior art reference teaches an apparatus for screening, sensing, or diagnosing disease states by obtaining a plurality of electrical impedance data measurements in organized patterns from two anatomically homologous body regions, one of which may be affected by disease. One subset of the data so obtained is processed and analyzed after structuring the data values as elements of an n.times.n impedance matrix, and deriving from these matrices their eigenvalues and eigenvectors. The matrices and their derivatives are then related by their characteristics to normal or disease states. In this patent, data are obtained from two anatomically homologous body regions, one of which may be affected by disease. Differences in the electrical properties of the two homologous body parts could signal disease. To the extent effective, this apparatus is limited in its functionality. Comparison of two anatomically homologous body regions for identifying results may not be applicable in those cases where person is disabled or handicapped. Plus one of the body parts may be affected by the decease less than the other body part and the results of the test will reveal difference between both parts thereby not providing clear picture for the purposes of diagnostics of the human health condition.

Alluding to the above, another published international patent application, PCT/CA01/01788, discloses a breast electrode array for diagnosing the presence of a disease state in a living organism, wherein the electrode array comprises a flexible body, a plurality of flexible arms extending from the body, and a plurality of electrodes provided by the plurality of flexible arms, wherein the electrodes are arranged on the arms to obtain impedance measurements between respective electrodes. In one embodiment, the plurality of flexible arms are spaced around the flexible body and are provided with electrode pairs, which can be used to make tetrapolar impedance measurement

Still another prior art reference, such as U.S. Pat. No. 6,434,422 to Tomoda et. al, for example, teaches a method and apparatus for judging a recovery of physical condition of a puerperal woman, wherein a bioelectric characteristic of the puerperal woman is measured and a value thereof is stored, and the recovery of her physical condition is judged based on the variation thereof. Further, the bioelectric characteristic of the woman in non-pregnancy period or at an early stage of her pregnancy period and the bioelectric characteristic of the puerperal woman are measured, and the recovery of her physical condition after delivery is judged based on the comparison between these bioelectric characteristics. The method and apparatus taught by the U.S. Pat. No. 6,434,422 to Tomoda et. al. have a very limited application because the technology disclosed therein is limited to judging a recovery of physical condition of the puerperal woman.

Yet another prior art reference developed by Medi. L.D. Corporation teaches a device for performing a functional medical diagnosis of the human body. The device permits to carry out of a health check by recording six skin zones on the human body. The device includes a housing, a connector cable, electrodes, and a computer. Although, it is advertised that the device is suitable for a rapid diagnosis and for medical follow-up for the medical and paramedical professions, the device requires multiple elements and necessity to connect these elements to six parts on the human body.

Alluding to the above, there numerous other traditional methods of instrumentation tests of physiological state of the human that include electroencephalography, ultrasound investigation, computer tomography and MR imaging etc. to the extend effective in some respect, there exist an area of continuous research and development for new diagnostic methods for the more efficient assessment of the most fine homeostasis processes.

For example, correlation between violation of a body functions and pathology of its separate cells, in particular, blood cells, is a known fact. Therefore, any disease of a body changes metabolism in cells with initiation of the processes of functional restructuring of cells and variation of emission spectra of proper EMF (electromagnetic fields) of cells. At the same time, such points, zones and areas are non-linear systems in electrophysical interpretation. It means that if EMF is applied to biologically active cell, or to a reflexogenic zone, a process of EMF interaction with proper EMF of the point, zone, or area takes place. Such interaction results in modulation of the external EMP at the emission frequency of biologically active cell. Separation of such frequency from the spectrum of modulated reflected wave and its analysis (amplitude-frequency) give the possibility to obtain the information on the state of a body and to assess the response to the external action on the body, in particular, in respect of physical fields.

Alluding to the above, the power of a cell signal is 10-12 Vt/m2, which considerably decreases the possibility of its direct registration by the modern radio-physical measurement devices.

Unfortunately, none of the aforementioned computer diagnostic systems is capable or equipped enough to provide full and comprehensive information on the health status of the human. Therefore, it is necessary to consult either a specialist or to resort to an instrument test of one or other specific organ or a system of the human.

Hence, there is a need for an improved method and system for non-invasive diagnostic of the human and correction of the condition of the human within a short period of time.

### SUMMARY OF THE INVENTION

An apparatus of the present invention is used for a non-invasive diagnostics and correction of humans and animals. The apparatus includes a computer, a reference database of the computer. The apparatus also a reversible sensing electrode device adaptable for retention by a human for receiving information about biological active cells of the human as the information is received through spectral signals. The reversible sensing electrode device is further defined by a housing having a first distal end and a second distal end and a hollow portion defined between the first and second distal ends. A pair of elements are attached to the housing. Each element is further defined by a plate having a semi-circular configuration. Each plate is formed from a metallic material. The plates are spaced from one another and do not present a direct contact with one another. Each plate is directly connected to a pair of electrodes. The housing is formed from a non-metallic material.

A connector element is disposed in the first distal end and is electrically connected to the pair of electrodes disposed inside the hollow portion. The connector presents a direct contact with the pair of elements, i.e. plates of semi-circular cross section to receive information about the biological active cells and transform the information about the biological active cells as the information is received through spectral signals to an audio processor cooperable with the reversible sensing electrode device. The audio processor receives the spectral signals from the reversible sensing electrode device and transforms the spectral signals to the digital codes. The audio processor is cooperable with the reversible sensing electrode device for receiving the spectral signals therefrom and transforming the spectral signals to digital codes is further defined by a VORTEX 2 PC I processor. A functional unit of the apparatus transforms the spectral signal through a wavelet transformation and compares the signals presented in the digital codes with the reference database and analysis of wavelet-transformed signal.

A computer software for analog-digital transformation of the spectral signals received from the reversible sensing electrode device and generation of resulting signal received by the reversible sensing electrode device in analog form performed by the audio processor thereby allowing the apparatus to function in diagnostic mode and corrective mode. When in diagnostic mode, the information reading from the biologically active cells is performed after pulse (0.5 s) influence on the biologically active cells is applied by electromagnetic fields with the frequency of at least one of 50.3 GHz and up to 50.3 GHz and power at least one of up to 10 μW/cm².

When correction mode is in effect, influence on the biologically active cells is performed by the electromagnetic field with the carrier frequency of at least one of 50.3 GHz and up to 50.3 GHz and applying a modulation frequency determined in accordance with formula ω_{H}=2ω₀/n [10], where ω₀ is the oscillator natural frequency, n is integer for cellular constructions, the modulation frequency is determined in accordance with formula

$${ω}_{0} = {U ( \frac{{π}^{2} {n}^{2} ρ {M}^{2}}{{l}^{2} {ρ}_{0} h ( 1 - {M}^{2} )} )}^{1 / 3} , n = 1 , 2 , …$$

where U is near membrane fluid flow; M is mach number; a is acoustic waves propagation velocity in a membrane of the biological active cell; l is a thickness and length of the membrane; ρ is a density of the fluid flow density; and ρ₀ is a lipid layer density of the membrane.

The apparatus of the present invention includes other functional components such as a unit of wavelet transformation of the signal MATLAB 6.0, a unit of comparison and analysis wavelet-converted or transformed signal with a reference database, a unit of synchronized scale, a unit of reference database, an imaging unit, a unit of correction and therapy, a unit of resulting signal, a unit of feedback and reversed communication, and a unit of recordation of reference data.

The reference database of the computer is further defined by a program that includes information about all organs and conditions of the human wherein the information is divided into separate groups identifying age, gender of the human, numbers of the biological active cells of each organ and other parts of the human and percentile of each biological active cells correlated to active range of the biological active cells and non-active range of the biological active cell.

An advantage of the present invention is to provide an effective method of the diagnostics and treatment systems for various illnesses of a living organism.

Another advantage of the present invention is to provide an apparatus and method adaptable to reveal diseases before their clinical display, including tumor and other pathological processes through non-invasive application.

Still another advantage of the present invention is to provide an apparatus and method adaptable to reveal activators of disease and exact area of their localization.

Still another advantage of the present invention is to provide an apparatus and method adaptable to reveal virus and bacteriological diseases and allergens.

Still another advantage of the present invention is to provide an apparatus and method adaptable to reveal and neutralize foreign objects in an organism including poisons, toxins, psychotropic substances, infections.

Still another advantage of the present invention is to provide an apparatus and method adaptable to remove a pressure and perfectly to stop painful syndromes, such as headaches of any degree of prescription.

Still another advantage of the present invention is to provide an apparatus and method adaptable to quickly and successfully to make selection of necessary medical preparations or target bio-proof-readers, food additives, grasses, various kinds of diets and techniques of treatment in strict conformity with specific features of an organism of each patient.

Still another advantage of the present invention is to provide an apparatus and method adaptable to effectively estimate influence of various kinds of diets, medicines, household chemical goods, building materials and means of agro chemistry.

Still another advantage of the present invention is to provide an apparatus and method adaptable to perform diagnostics and correction of the human within a short period of time.

Other advantages and meritorious features of this invention will be more fully understood from the following description of the preferred embodiment, the appended claims, and the drawings; a brief description of which follows.

### BRIEF DESCRIPTION OF THE DRAWINGS

Other advantages of the present invention will be readily appreciated as the same becomes better understood by reference to the following detailed description when considered in connection with the accompanying drawings wherein:

FIGS. 1 illustrates a general view of an apparatus of the present invention;

FIG. 2 illustrates a schematic view of the components of the present invention;

FIG. 3 illustrates a cross sectional view of a reversible sensing electrode device;

FIG. 4 illustrates a cross sectional view of an element, i.e. plate of semi-circular cross section plate disposed inside the reversible sensing electrode device;

FIG. 5 shows a partially cross sectional view of the housing of the reversible sensing electrode device;

FIG. 6 illustrates a biologically active cell presenting an active regions and a non active regions;

FIG. 7A illustrates a schematic view of a graph showing a radiation spectrum of a biologically active cell; and

FIG. 7B illustrates a schematic view of a graph showing a radiation spectrum of a biologically active cell having a pathology.

### DETAILED DESCRIPTION OF THE INVENTION

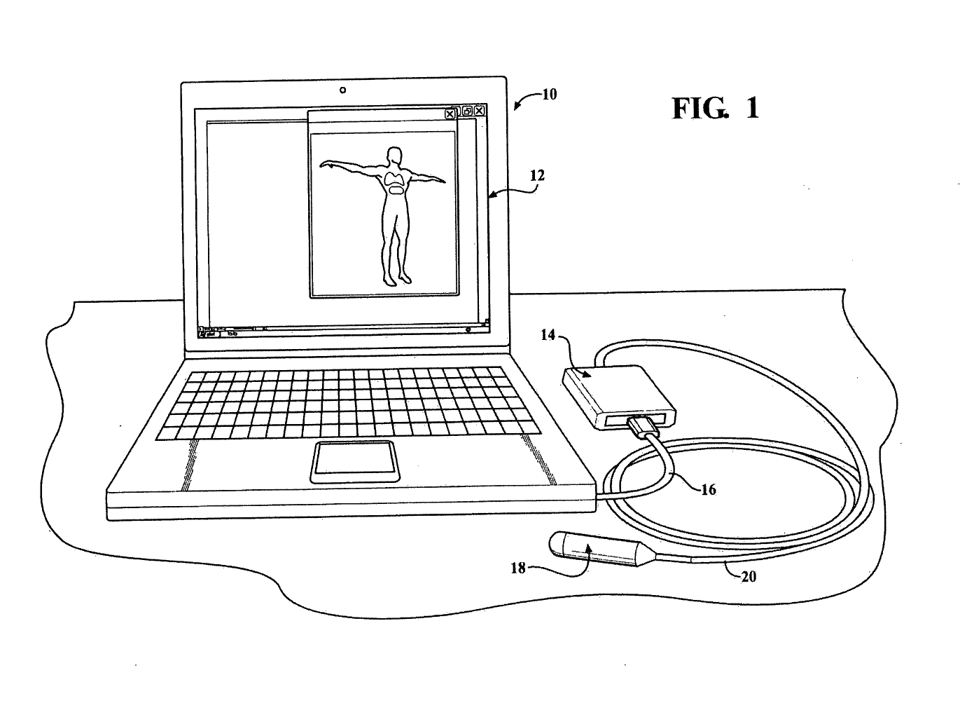

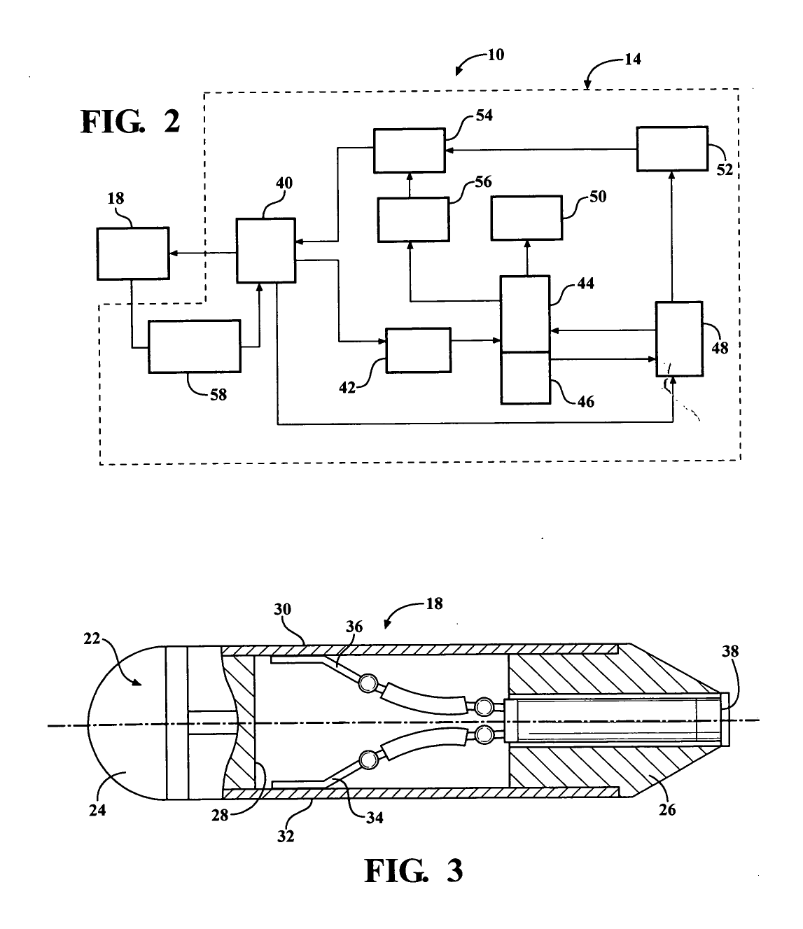

Referring to the Figures, wherein like numerals indicate like or corresponding parts throughout the several views, an apparatus of the present invention is generally shown at 10 in FIGS. 1 and 2. The apparatus 10 is used for medical purposes, which allow quick reading of information from an object, such as a human being or animal, and in order to provide expert analysis of the condition of the human being or the animal, and then detecting of affected cells and correction of the cells in order to bring an organism of the human or the animal into a normal or healthy state. The examination and correction may take place in both medical-diagnostics centers, laboratories, and in the field conditions, including in the places of emergency-related rescue operations. The apparatus 10 utilizes the new approaches to diagnosing and release of living organisms from pathological conditions caused by the influence of negative external and internal factors.

By the way of background, all auto-oscillation systems of an organism (a human or a mammal) are hard-mode systems when oscillations may build-up only with certain threshold amplitude. Biological object, such as a cell or biologically active cell, generally shown at 1 in FIG. 6, response to external oscillations of environment in the vicinity or range of f_{H}=2f_{O}/n. As appreciate by those skilled in the art, any object in the universe may acquire, store and convey information. A mammal such as a human or animal, as any living biological object in the universe, may also process and use information. In particular, the human and the animals have their proper oscillations. These are just wave processes with their inherent frequency and amplitude characteristics. Using certain methods, scientists took readings of such characteristics both of pathological organs with “incorrect” oscillations and of normally functioning organs with “correct” oscillations.

A special mode of display of test results arranges the results of diagnosing organs and systems of a body according to a certain scheme, depending on degree of their affection and severity of an illness, thereby identifying presence of pathologies and neoplasms in the body. The apparatus 10 allows simultaneous comparison of diagnosing results of the human or animal for different periods, as well as to observe dynamics of changes in the human or animal in digital or graphic display.

The operation of the apparatus 10 is based on the currently known theory that any object in the universe may acquire, store and convey information. A man, as any living biological object in the universe, may also process and use information. In case of theoretical study of the lower (resonance-wave) informational level as wave and field oscillations of elementary particles, their relations and interaction manifest themselves as resonance-wave processes with certain frequency characteristics and wave length. Interference of such processes on the follow-up level creates new indices of information frequencies on the same level. The integrated resonance-wave processes of elementary particle level create spin fields, and as a result of further interference determine the informational components of each next level. In this case, the level information in unaffected by informational substrate (either material or non-material), but depends only on the informational capacity of the level.

Thus, each level has its own frequency, different from others, of auto-oscillations synchronized in the organism into a single auto-oscillation information-and-frequency process. The informational level interaction results in that every organ is functioning in a certain frequency range. Therefore any organism disease changes the metabolic processes behavior in the cells that in its turn initiates the cells functional changes and variations of the cell proper electromagnetic fields (EMF) spectra. As shown in FIG. 6, for example, the cell 1 includes a normal, i.e. healthy section 2 and abnormal sections 4 and 6, wherein each of these sections 2, 4, and 6, presents “incorrect” oscillations as compared to “correct” oscillations of normally functioning organs. Such frequencies are called biologically effective frequencies or the resonance frequencies.

For example, existence of the biologically effective frequencies can be explained by forced resonance or the parametric one with the proper frequencies of the organism micro-resonator (molecules, blood elements, cells, membranes and DNA). Information on the proper frequencies of given system or organ allows to determine the biologically effective for this organism frequencies of external medium. The natural frequency values depend on the oscillator dimensions L (e.g. heart). All the organism self-sustained oscillation systems are the systems with the hysteresis when the oscillations can rise only after some threshold amplitude. The biological objects response to external medium oscillations appears in the near neighborhood of values ω_{H}=2ω₀/n; this neighborhood width is greater when the parameter change amplitude is greater too.

One of the most important the biologically active cell 1 morphofunctional formations are the mast cells containing up to 80% of water and with considerably higher density than in the skin “inactive” zones reactive therapeutic levels are power levels within 10⁻¹⁴-10⁻¹⁰ W/m². At the same time the water state in the biologically active cell 1 structures can essentially influence on the cell signals amplifying or attenuation. The cell aqueous solution represents the system which is able for uniform integration of the various physical and chemical effects including the magnetic, eclectic and electromagnetic fields influence.

A test signal is sent to the biologically active cell 1 of a certain meridian (j), whereas the information on response of the r biologically active cell 1 of the respective organ or system in taken from other biologically active cell 1 of this (j) meridians or of the meridians connected with it (j+k, . . . , N). Besides, frequency f, amplitude A and form φ of signal vary in test signal in the manual and/or automatic mode. The possibility of synthesis of test signals (via selection of f, A, and φ parameters) adequate to the signal model with the parameters of a healthy person is stipulated.

Referring back to FIGS. 1 and 2, the apparatus 10 of the present invention is used for a non-invasive diagnostics and correction of the humans and the animals. The apparatus 10 includes a computer, generally indicated at 12. Any time of the computer 12 may be used in application of this invention without limiting the scope of it. A functional component, generally indicated at 14, is operably communicated with the computed 12. The connection between the functional component 14 and the computer may be wireless as well as direct through conventional means of connections such as wires and cords 16. The apparatus 10 also includes a reversible sensing electrode device (the sensor), generally indicated at 18. The connection between the functional component 14 and the sensor 18 may be wireless as well as direct through conventional means of connections such as wires and cords 20. The sensor 18 is adaptable for retention by the human or the animal for receiving information about biologically active cells as the information is received through spectral signals. The sensor 18 may also be placed at different parts of the body of the human or the animal to obtain the signals.

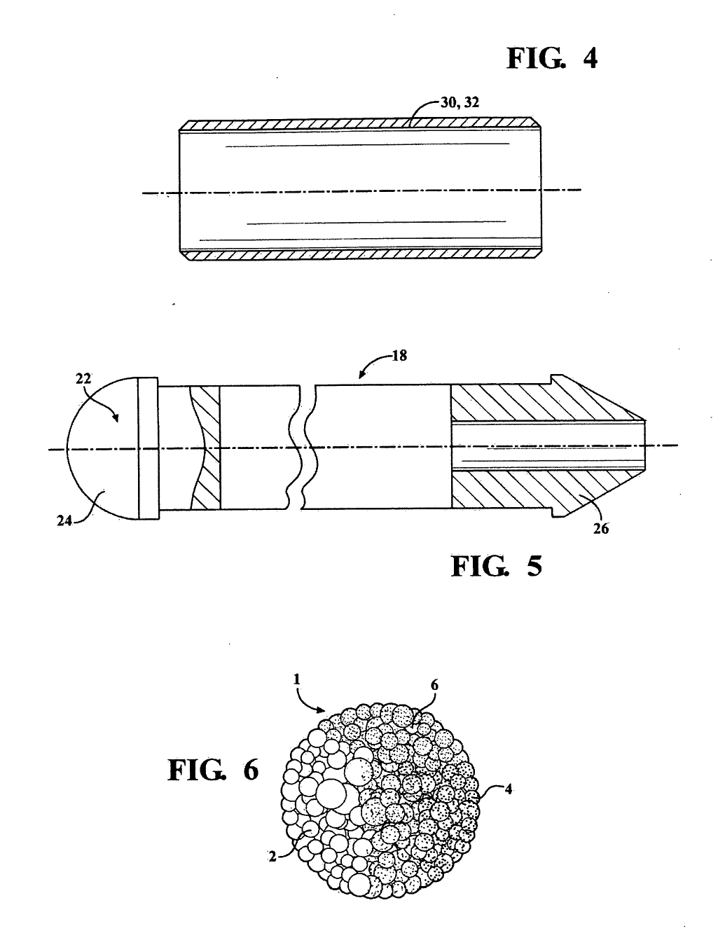

Referring to FIGS. 3 through 5, the sensor 18 is further defined by a housing, generally indicated at 22, having a first distal end 24 and a second distal end 26 and a hollow portion 28 defined between the first and second distal ends 24 and 26. The housing 22 is formed from a non-metallic material such plastic, wood, or any other type of a polymeric material. A pair of elements 30 and 32 is attached to the housing 22. Each element 30, 32 is further defined by a plate having a semi-circular configuration. Each plate is formed from a metallic material. The plates are spaced from one another and do not present a direct contact with one another. A semi-circular cross section of the elements 30, 32 is not intended to limit the scope of the present invention. The housing 22 and the elements 30, 32 may present a non-circular, i.e. a square configuration without limiting the scope of the present invention.

Each plate 30, 32 is directly connected to a pair of electrodes 34, 36. A connector element 38 is disposed in the second distal end 26 and is electrically connected to the pair of electrodes 34, 36 disposed inside the hollow portion 28. The connector element 38 presents a direct contact with the pair of elements 30, 32, i.e. plates of semi-circular cross section to receive information through spectral signals about the biologically active cells and transform the information about the biological active cells as the information is received through the functional component 14 details of which will discussed herebelow.

As best shown in FIG. 2, the functional component 14 includes an audio processor 40 cooperable with the sensor 18. The audio processor 40 receives the spectral signals from the reversible sensing electrode device 18 and transforming the spectral signals to the digital codes. The audio processor 40 is a VORTEX 2 PC I processor. Other processors may be used without limiting the scope of the present invention. The audio processor 40 transforms the spectral signal through a wavelet transformation by comparing the signals presented in the digital codes with the reference database and analysis of wavelet-transformed signal. A computer software for analog-digital transformation of the spectral signals received from the reversible sensing electrode device 18 and generation of resulting signal received by the reversible sensing electrode device 18 in analog form performed by the audio processor 40 thereby allowing the apparatus 10 to function in diagnostic mode and corrective mode.

The functional component 14 includes a unit of wavelet transformation of the signal MATLAB 6.0 42, a unit of comparison and analysis wavelet-converted or transformed signal with a unit of comparison of information 44, a unit of synchronized scale 46, a unit of reference database 48, an imaging unit 50, a unit of correction and therapy 52, a unit of resulting signal 54, a unit of feedback and reversed communication 56, and a unit of recordation of reference data 58.

The unit of comparison and analysis wavelet-converted or transformed signal with the unit of comparison of information 44 includes a software program that includes information about all organs and conditions of the human wherein the information is divided into separate groups identifying age, gender of the human and the animal, numbers of the biological active cells of each organ and other parts of the human and the animal percentile of each biological active cells correlated to active range of the biological active cells and non-active range of the biological active cell.

The software program also contains information about various pathologic processes taking into account the age and sex of the mammals. The software also stores the information about the spectral characteristics of each organ of the mammal and each biologically active cell having its own specific frequencies. These frequencies can be displayed on the screen of the computer 12 in the form of spectrum or other graphical illustrations reflecting the condition of the organs and tissues of the mammals.

When in diagnostic mode, the information reading from the biologically active cells is performed after pulse (0.5 s) influence on the biologically active cells is applied by electromagnetic fields with the frequency of at least one of 50.3 GHz and up to 50.3 GHz and power at least one of up to 10 μW/cm². When correction mode is in effect, influence on the biologically active cells is performed by the electromagnetic field with the carrier frequency of at least one of 50.3 GHz and up to 50.3 GHz and applying a modulation frequency determined in accordance with formula ω_{H}=2ω₀/n [10], where ω₀ is the oscillator natural frequency, n is integer for cellular constructions, the modulation frequency is determined in accordance with formula

$${ω}_{0} = {U ( \frac{{π}^{2} {n}^{2} ρ {M}^{2}}{{l}^{2} {ρ}_{0} h ( 1 - {M}^{2} )} )}^{1 / 3} , n = 1 , 2 , …$$

where U is near membrane fluid flow; M is mach number; a is acoustic waves propagation velocity in a membrane of the biological active cell; l is a thickness and length of the membrane; ρ is a density of the fluid flow density; and ρ₀ is a lipid layer density of the membrane.

Alluding to the above, as the apparatus 10 is in operational mode to perform the diagnostics of the human or the animal, the sensor 14 through biologically active points on the human body takes the spectrum of electromagnetic radiation from the human or the animal, and passes the spectrum of electromagnetic radiation from the human or the animal through an electrical signal to the audio processor 40. The audio processor 40 transforms the electric signals carrying the information from analogue mode or format to a digital mode or format. The converted signal is then fed into the unit of wavelet transformation of the signal MATLAB 6.0 42.

After the converted signal is processed, it then passes to the unit of reference database 48 through the unit of comparison of information 44 and the unit of synchronized scale 46. The unit of reference database 48 and the unit of synchronized scale 46 are operably connected with one another for the purpose phase synchronization wavelet spectral characteristics of the biologically active points of the spectrum wavelet reference database. The results of the comparison and analysis of from the unit of comparison of information 44 are received in the imaging unit 50 and then are displayed on a screen of the computer 12 in the form of diagnosis of the organism.

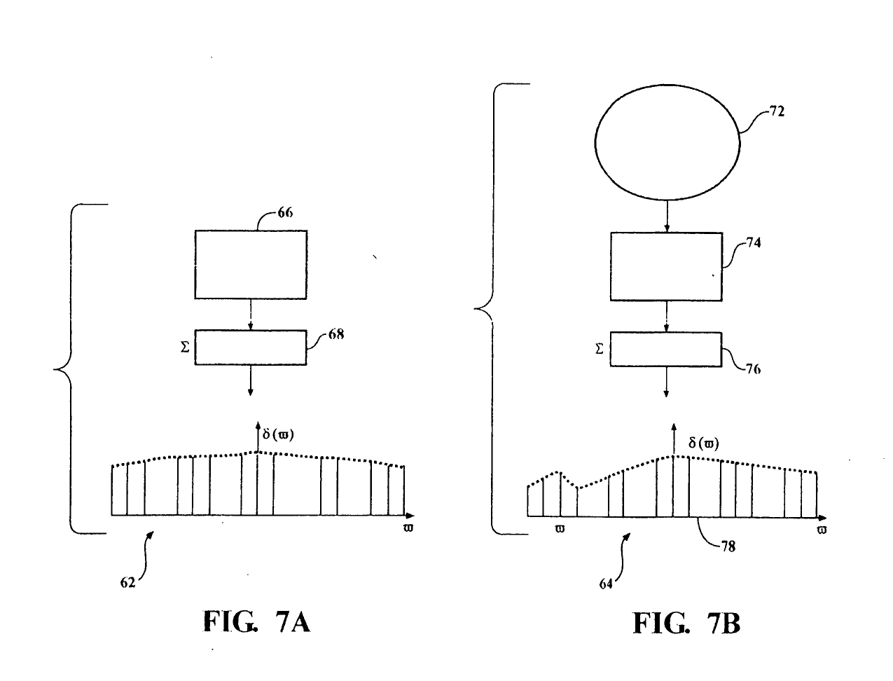

As best illustrated in FIGS. 7A and 7B, FIG. 7A illustrates a schematic view of a graph showing a radiation spectrum of a biologically active cell, as generally shown at 62. FIG. 7B illustrates a schematic view of a graph showing a radiation spectrum of a biologically active cell having a pathology, generally shown at 64. Referring back to FIG. 7A, a human organism, i.e. a body, is shown at 66. The organism 66 is comprised of a plurality of cells 68. Each cell 68 emits signals. The sensor 14 through biologically active cells 68 of the human body 66 takes the spectrum of electromagnetic radiation from the body 66, and passes the spectrum of electromagnetic radiation from the body 66 through the electrical signal to the audio processor 40.

As shown in FIG. 7B, a human organism, i.e. a body, is shown at 74. The organism 74 is comprised of a plurality of cells 76. Each cell 76 emits signals. The sensor 14 through biologically active cells 76 of the human body 74 takes the spectrum of electromagnetic radiation from the body 74, and passes the spectrum of electromagnetic radiation from the body 74 through the electrical signal to the audio processor 40. FIG. 7B shows a graph 78 depicting an abnormal or not healthy spectrum of radiation emitted from the cell 76 as the body 74 and negatively affected by decease or by other pathology 72.

After receiving the diagnosis, the physician or another health care professional, determines the effective and corrective and medical bio-physical standards of the unit of reference database 48 and communicates with the unit of correction and therapy 52. The unit of correction and therapy 52 send a signal to the unit of resulting signal 54, which is cooperable with the unit of feedback and reversed communication 56 thereby correcting and adjusting the range of the resulting signal required to affect and impact the cell 1. After the resulting signal is corrected at the unit of resulting signal 54, its then transferred to the audio processor 40. The audio processor 40 thereby acts as a generator of analog electrical impulses. The audio processor sends an electric pulse in analog form the sensor 14 reverse sensor, wherein the electrical pulse in the analog form is converted into electromagnetic radiation. As the electrical pulse is converted into electromagnetic radiation and is in its within range of biologically active points, the electromagnetic radiation through the sensor 14 starts to affect these acupressure points, realizing that influence of the correction of the spectra and therapy of the body and treat affected areas 4 and 6 of the cell 1.

Alluding to the above, as the electromagnetic fields are applied to biologically active cell 1, or to a reflexogenic zone, a process of electromagnetic fields interaction with proper electromagnetic fields of the point, zone, or area takes place. Such interaction results in modulation of the external electromagnetic fields at the emission frequency of the biologically active cell 1. Separation of such frequency from the spectrum of modulated reflected wave and its analysis (amplitude-frequency) allows to obtain the information on the state of a body and to assess the response to the external action on the body, in particular, in respect of physical fields.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. An apparatus for non-invasive diagnostics and correction of a condition of a human, said apparatus comprising;
a computer,
a reference database of said computer including information about all organs and conditions of the human wherein the information is grouped into categories and correlations of said categories where each of said categories includes age, gender of the mammal, numbers of the biological active cells of each organ and other parts of the mammal and percentile of each biological active cells correlated to an active range of the biological active cells and non-active range of the biologically active cell;
a reversible sensing electrode device engaged by the human for receiving information about the biologically active cells as the information received through spectral signals extending from the biologically active cells,
an audio processor cooperable with said reversible sensing electrode device for receiving the spectral signals and transforming the spectral signals to digital codes,
a functional unit presenting a computer software configured to receive the information from said reversible sensing electrode device to identify percentile of each biological active cells correlated to said active range of the biological active cells and said non-active range of the biologically active cell and impact non-active biological cells through electromagnetic waves thereby increasing the ratio of the biologically active cells in relationship to the non-active biological cells, wherein said functional unit for transforming the spectral signal through a wavelet transformation is cooperable with said reference database of said computer and said computer software for analog-digital transformation of the spectral signals received from said reversible sensing electrode device for comparing the signals presented in the digital codes with said reference database having said program that includes information about all organs and conditions of the mammal wherein the information is divided into separate groups identifying age, gender of the mammal, numbers of the biological active cells of each organ and other parts of the mammal and percentile of each biological active cells correlated to active range of the biological active cells and non-active range of the biological active cell.

2. (canceled)

3. An apparatus as set forth in claim 1, wherein said reversible sensing electrode device is further defined by a housing having a first end and a second end and a hollow portion defined between said first and second ends, a pair of elements attached to said housing, a connector element disposed in said first distal-end, and a pair of electrodes disposed inside said hollow portion and presenting a direct contact with said pair of elements to receive information about the biological active cells and transform the information about the biological active cells as the information is received through spectral signals to said audio processor cooperable with said reversible sensing electrode device.

4. An apparatus as set forth in claim 1, wherein said housing is formed from a non-metallic material.

5. An apparatus as set forth in claim 1, wherein each of said elements is further defined by a plate having a semi-circular configuration and is formed from a metallic material.

6. An apparatus as set forth in claim 1, wherein said plates are spaced from one another and do not present a direct contact with one another.

7. An apparatus as set forth in claim 1, wherein each of said plates is directly connected to each of said electrodes.

8. (canceled)

9. An apparatus as set forth in claim 1, wherein said audio processor cooperable with said reversible sensing electrode device for receiving the spectral signals therefrom and transforming the spectral signals to digital codes is further defined by a processor.

10. (canceled)

11. An apparatus as set forth in claim 1, wherein said functional unit includes a unit of wavelet transformation of the signal.

12. An apparatus as set forth in claim 1, wherein said functional unit includes a unit of comparison and analysis of a wavelet-converted or transformed signal with a reference database.

13. An apparatus as set forth in claim 1, wherein said functional unit includes a unit of synchronized scale.

14. (canceled)

15. An apparatus as set forth in claim 2, including a display unit.

16. An apparatus as set forth in claim 1, wherein said functional unit includes a unit of correction and therapy.

17. An apparatus as set forth in claim 1, wherein said functional unit includes a unit of resulting signal.

18. An apparatus as set forth in claim 1, wherein said functional unit includes a unit of feedback and reversed communication.

19. (canceled)
